(19) United States
(12) Patent Application Publication
(10) Pub. No.: US 2024/0058404 A1
(43) Pub. Date: Feb. 22, 2024

(54) IMPROVED METHODS TO ENHANCE GASTROINTESTINAL HEALTH

(71) Applicant: ARBIOM SAS, Paris (FR)

(72) Inventor: Ricardo EKMAY, Morrisville, NC (US)

(21) Appl. No.: 18/270,092

(22) PCT Filed: Jan. 5, 2022
(86) PCT No.: PCT/EP2022/050151

§ 371 Date: Jun. 28, 2023

Related U.S. Application Data
(63) Continuation of application No. PCT/EP2021/050088, filed on Jan. 5, 2021.

(51) Int. Cl. A61K 36/06; A61K 35/747; A61K 36/05

(52) U.S. Cl. CPC A61K 36/06; A61K 35/747; A61K 36/05; C12N 1/12

(57) ABSTRACT

The present invention relates to a composition for use in a method of preventing and/or treating a gastrointestinal disorder, preferably gastrointestinal disease, wherein said composition comprises microorganisms cultivated on or with lignocellulosic hydrolysate, optionally further comprises lignocellulosic hydrolysate and/or a carrier. Furthermore, the present invention relates to a method of preparing a composition. The present invention also relates to a method of preventing or treating a gastrointestinal disorder, a method of enhancing gastrointestinal health, and a method of reducing gastrointestinal permeability in a patient.

### FIELD OF THE INVENTION

The present invention relates to a composition for use in a method of preventing and/or treating a gastrointestinal disorder, preferably gastrointestinal disease, wherein said composition comprises microorganisms cultivated on or with lignocellulosic hydrolysate, optionally further comprises lignocellulosic hydrolysate and/or a carrier. Furthermore, the present invention relates to a method of preparing a composition. The present invention also relates to a method of preventing or treating a gastrointestinal disorder, a method of enhancing gastrointestinal health, and a method of reducing gastrointestinal permeability in a patient.

### BACKGROUND OF THE INVENTION

Humans and animals may suffer from conditions such as Crohn's disease, ulcerative colitis, irritable bowel syndrome, celiac disease, post-weaning diarrhea, colibacillosis, porcine epidemic diarrhea virus, necrotic enteritis, and soybean meal-induced enteritis, among others.

Gastrointestinal permeability is a condition colloquially known as “leaky gut syndrome”. Gastrointestinal permeability may initiate a pro-inflammatory cascade leading to systemic inflammation, reduced nutrient absorption, and impaired health. This cascade is mediated through various chemical messengers, e.g. cytokines, whose control is critical to prevent systemic inflammation [1, 2]. Increased gastrointestinal permeability is associated with development of Crohn's disease, irritable bowel syndrome, celiac disease, and infectious diarrhea. Increased gastrointestinal permeability may occur through the consumption of certain ingredients, for example, vegetable proteins such as wheat or soy; through the colonization of certain live microorganisms, for example, Escherichia coli, Clostridium spp.; and through certain nutrient deficiencies, for example, zinc.

Increased gastrointestinal permeability and the resulting impairment of gastrointestinal health may result in increased morbidity and mortality resulting in sub-optimal growth, feed-efficiency, and economic returns in commercially important livestock species. Furthermore, impaired gastrointestinal health may result in general morbidity in companion animal species. Increased gastrointestinal permeability is also involved in various human gastrointestinal disorders.

It has been proposed to add yeast and other microorganisms as probiotica to improve gastrointestinal health [3]. For example, inclusion of yeast and yeast derivatives as probiotic and prebiotic-like substances, respectively, in animal feed has been proposed to have positive associations with growth performance. However, there is a need for means to ameliorate and/or reduce gastrointestinal permeability. Furthermore, there is a need to provide methods of preventing or treating gastrointestinal disorders involving increased gastrointestinal permeability. There is also the need for means, such as compositions, allowing enhancing gastrointestinal health, particularly allowing reducing gastrointestinal permeability. Furthermore, there is a need to provide enhanced compositions, food formulations, and/or feed formulations ameliorating gastrointestinal permeability.

### SUMMARY OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the present invention relates to a composition for use in a method of preventing and/or treating a gastrointestinal disorder, preferably gastrointestinal disease, wherein said composition comprises microorganisms cultivated on or with lignocellulosic hydrolysate, optionally further comprises lignocellulosic hydrolysate and/or a carrier.

In one embodiment, said gastrointestinal disorder is selected from increased gastrointestinal permeability, leaky gut syndrome, inflammatory bowel disease, Crohn's disease, ulcerative colitis, irritable bowel syndrome, necrotic enteritis, colibacillosis, diarrhea/scours, porcine epidemic diarrhea virus, soybean meal induced enteritis, transient soy hypersensitivity, celiac disease, post-weaning diarrhea, necrotic enteritis, diabetes, rheumatoid arthritis, spondyloarthropathies, schizophrenia, cancer, fatty liver disease, atopy, and allergies.

In one embodiment, said gastrointestinal disorder is selected from increased gastrointestinal permeability, leaky gut syndrome, inflammatory bowel disease, Crohn's disease, ulcerative colitis, irritable bowel syndrome, necrotic enteritis, colibacillosis, diarrhea/scours, porcine epidemic diarrhea virus, soybean meal induced enteritis, transient soy hypersensitivity, celiac disease, post-weaning diarrhea, and necrotic enteritis; preferably selected from leaky gut syndrome, Crohn's disease, irritable bowel syndrome, diarrhea/scours, and celiac disease.

In one embodiment, said microorganisms are selected from yeast, fungi, bacteria, microalgae, and combinations thereof. In a preferred embodiment, said microorganisms are selected from yeast.

In one embodiment, said yeast is selected from Candida sp., Saccharomyces sp., Kluyveromyces sp., Cyberlindnera sp., and combinations thereof; and/or
said fungi are selected from Aspergillus sp., Paecilomyces sp., Pleurotus sp., Trichoderma sp., and combinations thereof; and/or
said bacteria are selected from Bacillus sp. and Lactobacillus sp., and combinations thereof; and/or
said microalgae are selected from Spirulina sp. and Chlorella sp., and combinations thereof.

In one embodiment, said yeast is torula yeast. In one embodiment, torula yeast is Cyberlindnera jadinii.

In one embodiment, said microorganisms have been subjected to a cultivation on or with lignocellulosic hydrolysate for a period of 0.1 to 600 h, preferably 1 to 350 h, at a temperature of 15 to 60° C., preferably 20 to 45° C., optionally as a batch, fed-batch, and/or continuous cultivation.

In one embodiment, said lignocellulosic hydrolysate is derived from biomass selected from forestry products and residues, e.g. wood and sawdust, agricultural crops, agricultural products, agricultural residues, e.g. corn, sugar cane, bagasse, and stover, crops e.g. energy crops, and waste products, e.g. food waste, solid paper waste, and spent liquors.

In one embodiment, said lignocellulosic hydrolysate is carbohydrate-rich lignocellulosic hydrolysate; and/or
said lignocellulosic hydrolysate is prepared using any of physical, chemical, physicochemical, and/or biological treatment, preferably prepared from biomass via biomass fractionation, chemical breakdown, enzymatic breakdown, physical breakdown e.g. steam explosion, or combinations thereof, more preferably prepared via biomass fractionation followed by an enzymatic hydrolysis of one or more of a fractionated biomass.

In one embodiment, in said method, said composition is administered to a patient in need thereof, wherein said patient is an animal, preferably a vertebrate or an invertebrate, or a human.

In one embodiment, said vertebrate is selected from a mammal, bird, and fish,
wherein, preferably,
said mammal is selected from a pig, dog, and cat, and/or
said bird is selected from a chicken, turkey, and duck, and/or
said fish is selected from salmon, trout, bass, bream, cod, catfish, and tilapia;
and/or wherein said invertebrate is selected from a shrimp, lobster, crab, octopus, squid, oyster, clam, and mussel.

In one embodiment, in said method, said composition is administered to said patient via oral administration, optionally jointly with food intake.

In one embodiment, said composition comprises a carrier selected from starches e.g. corn starch, dextrins, oligosaccharides, fibers e.g. cellulose, proteins e.g. soya, and oils e.g. vegetable oil.

In one embodiment, said composition is formulated in the form of a powder, granules, a tablet, a pill, a capsule, a dispersion, a solution, a suspension, an emulsion, a liquid, a liquid concentrate, an oil mixture, a bar, a stick, a food product, a food supplement, a drink, a food formulation, a feed formulation, or a lozenge.

In one embodiment, said composition is a food formulation or a feed formulation comprising said composition in an amount of 0.001-50% by weight, preferably 0.1-20% by weight, e.g. 0.1-10% by weight, optionally further comprising food and/or a beverage.

In a further aspect, the present invention relates to a method of preparing a composition, as defined above, comprising the following steps:
i) providing a microorganism, preferably selected from yeast, fungi, bacteria, microalgae, and combinations thereof, and a lignocellulosic hydrolysate;
ii) culturing said microorganism using said lignocellulosic hydrolysate as a medium, thereby obtaining a mixture of microorganism and lignocellulosic hydrolysate;
iii) optionally, separating said microorganism from said medium, e.g. by centrifugation,
iv) optionally, heat-inactivating said microorganism and/or said mixture,
v) optionally, drying said microorganism and/or said mixture, preferably to a moisture content of <10% by weight,
vi) optionally, adding a carrier to said microorganism and/or said mixture,
vii) obtaining a composition comprising said microorganism and/or said mixture, optionally further comprising said carrier.

In one embodiment, said providing said lignocellulosic hydrolysate comprises treating biomass, preferably using physical, chemical, physicochemical, and/or biological treatment, e.g. using
biomass fractionation,
preferably for a period of 0.5 to 360 min, preferably 1 to 120 min, at a temperature of 50 to 300° C., preferably 120 to 220° C., e.g. 120 to 216° C., and/or using any of physical, chemical, physicochemical, and biological treatment, or combinations thereof;
chemical breakdown,
preferably for a period of 0.5 to 360 min, preferably 5 to 60 min, at a temperature of 50 to 300° C., preferably 130 to 200° C., and/or using one or more chemicals, e.g. selected from acids, alkalis, ionic liquids, ammonia, and organic solvents;
enzymatic breakdown,
preferably for a period of 0.1 to 200 h, preferably 1 to 96 h, at a temperature of 20 to 80° C., preferably 40 to 60° C., and/or using a cellulose, hemicellulose, or a combination thereof; and/or
physical breakdown,
preferably for a period of 0.1 to 200 h, preferably 12 to 48 h, at a temperature of 20 to 80° C., preferably 48 to 55° C., and/or using any of shaking, stirring, swirling, crushing, agitating, or combinations thereof;
or combinations thereof.

In one embodiment, said providing said lignocellulosic hydrolysate, preferably said treating biomass, comprises a biomass fractionation followed by an enzymatic hydrolysis.

In one embodiment, said providing said lignocellulosic hydrolysate, preferably said treating biomass, comprises a biomass fractionation, thereby obtaining fractionated biomass, followed by an enzymatic hydrolysis of a fractionated biomass.

In one embodiment, said providing said lignocellulosic hydrolysate comprises preparing lignocellulosic hydrolysate.

In one embodiment, said culturing comprises a cultivation of said microorganism on or with said lignocellulosic hydrolysate for a period of 0.1 to 600 h, preferably 1 to 350 h, at a temperature of 15 to 60° C., preferably 20 to 45° C., optionally as a batch, fed-batch, and/or continuous cultivation.

In this aspect, said composition, said microorganism, said yeast, said fungi, said bacteria, said microalgae, said lignocellulosic hydrolysate, said culturing, said carrier, and said preparing lignocellulosic hydrolysate are as defined above.

In a further aspect, the present invention relates to a method of preventing or treating a gastrointestinal disorder, preferably gastrointestinal disease, wherein said method comprises administering a composition, as defined above, to a patient in need thereof.

In one embodiment, said disorder is selected from increased gastrointestinal permeability, leaky gut syndrome, inflammatory bowel disease, Crohn's disease, ulcerative colitis, irritable bowel syndrome, necrotic enteritis, colibacillosis, diarrhea/scours, porcine epidemic diarrhea virus, soybean meal induced enteritis, transient soy hypersensitivity, celiac disease, post-weaning diarrhea, necrotic enteritis, diabetes, rheumatoid arthritis, spondyloarthropathies, schizophrenia, cancer, fatty liver disease, atopy, and allergies.

In one embodiment, said administering comprises administering an effective amount of said composition to a patient in need thereof.

In one embodiment, said effective amount is in the range of from 0.001 to 50% by weight, preferably of from 0.1 to 20% by weight, of a total weight of a daily food consumption, and/or is in the range of 0.5-100 g/kg bodyweight per day, preferably 2-10 g/kg bodyweight per day, e.g. about 5.5 g/kg bodyweight per day.

In one embodiment, said patient is an animal, preferably a vertebrate or an invertebrate, or a human.

In one embodiment, said vertebrate is selected from a mammal, bird, and fish,
wherein, preferably,
said mammal is selected from a pig, dog, and cat, and/or
said bird is selected from a chicken, turkey, and duck, and/or
said fish is selected from salmon, trout, bass, bream, cod, catfish, and tilapia;
and/or wherein said invertebrate is selected from a shrimp, lobster, crab, octopus, squid, oyster, clam, and mussel.

In one embodiment, said composition is administered to said patient via oral administration, optionally jointly with food intake.

In this aspect, said gastrointestinal disorder, said administering, said effective amount, said composition, and said patient are as defined above.

In a further aspect, the present invention relates to a method of enhancing gastrointestinal health, wherein said method comprises administering a composition, as defined above, to a patient in need thereof.

In one embodiment, said administering comprises administering an effective amount of said composition to a patient in need thereof.

In one embodiment, said effective amount is in the range of from 0.001 to 50% by weight, preferably of from 0.1 to 20% by weight, of a total weight of a daily food consumption, and/or is in the range of 0.5-100 g/kg bodyweight per day, preferably 2-10 g/kg bodyweight per day, e.g. about 5.5 g/kg bodyweight per day.

In one embodiment, said patient is an animal, preferably a vertebrate or an invertebrate, or a human.

In one embodiment, said vertebrate is selected from a mammal, bird, and fish,
wherein, preferably,
said mammal is selected from a pig, dog, and cat, and/or
said bird is selected from a chicken, turkey, and duck, and/or
said fish is selected from salmon, trout, bass, bream, cod, catfish, and tilapia;
and/or wherein said invertebrate is selected from a shrimp, lobster, crab, octopus, squid, oyster, clam, and mussel.

In one embodiment, said composition is administered to said patient via oral administration, optionally jointly with food intake.

In this aspect, said administering, said effective amount, said composition, and said patient are as defined above.

In a further aspect, the present invention relates to a method of reducing gastrointestinal permeability in a patient, wherein said method comprises administering a composition, as defined above, to a patient in need thereof.

In one embodiment, said administering comprises administering an effective amount of said composition to a patient in need thereof.

In one embodiment, said effective amount is in the range of from 0.001 to 50% by weight, preferably of from 0.1 to 20% by weight, of a total weight of a daily food consumption, and/or is in the range of 0.5-100 g/kg bodyweight per day, preferably 2-10 g/kg bodyweight per day, e.g. about 5.5 g/kg bodyweight per day.

In one embodiment, said patient is an animal, preferably a vertebrate or an invertebrate, or a human.

In one embodiment, said vertebrate is selected from a mammal, bird, and fish,
wherein, preferably,
said mammal is selected from a pig, dog, and cat, and/or
said bird is selected from a chicken, turkey, and duck, and/or
said fish is selected from salmon, trout, bass, bream, cod, catfish, and tilapia;
and/or wherein said invertebrate is selected from a shrimp, lobster, crab, octopus, squid, oyster, clam, and mussel.

In one embodiment, said composition is administered to said patient via oral administration, optionally jointly with food intake.

In this aspect, said administering, said effective amount, said composition, and said patient are as defined above.

In a further aspect, the present invention relates to a use of a composition, as defined above, for the manufacture of a medicament, preferably a medicament for a gastrointestinal disorder.

In this aspect, said composition and said gastrointestinal disorder are as defined above.

In a further aspect, the present invention relates to a use of a composition, as defined above, for the manufacture of a food formulation or a feed formulation.

### DETAILED DESCRIPTION

It is an aim of the invention to provide means for ameliorating and/or reducing gastrointestinal permeability. Furthermore, the invention aims at providing methods of preventing or treating gastrointestinal disorders, such as gastrointestinal disorders involving increased gastrointestinal permeability. It is also an aim of the invention to provide means, such as compositions, allowing enhancing gastrointestinal health, particularly allowing reducing gastrointestinal permeability. Furthermore, it is an aim of the invention to provide enhanced compositions, food formulations, and/or feed formulations ameliorating gastrointestinal permeability. Furthermore, it is an aim of the invention to alleviate the symptoms of increased gastrointestinal permeability. Furthermore, it is an aim to provide enhanced food products, e.g. food formulations, and feed products, e.g. feed formulations, that enhance gastrointestinal health, particularly reduce gastrointestinal permeability.

This invention relates to new and improved methods of enhancing gastrointestinal health through reduction of gastrointestinal permeability and the resulting inflammatory response via the use of microorganisms cultivated on lignocellulosic hydrolysates, such as yeast and yeast-derived additives, which have been cultivated on lignocellulosic hydrolysates.

In one embodiment, administration of microorganisms such as, but not limited to, yeast and fractions thereof improves gastrointestinal health in humans and animals. In one embodiment, administration of microorganisms such as torula yeast cultivated on lignocellulosic hydrolysates, e.g. in the amount of 0.1-20% of total feed/food by weight, improves gastrointestinal health in humans and animals. In one embodiment, administration of microorganisms cultivated on lignocellulosic hydrolysates, e.g. in the amount of 0.1-20% of total feed/food by weight, improves gastrointestinal health in humans and animals, and such effect of improving gastrointestinal health, particularly reducing gastrointestinal permeability, is superior compared to an effect of microorganisms cultivated on a medium/substrate other than lignocellulosic hydrolysate. In one embodiment, the improvement in gastrointestinal health is greater when using yeast or other microorganisms which have been cultivated on lignocellulosic hydrolysates compared to when using yeast or other microorganisms which have not been cultivated on lignocellulosic hydrolysates. In one embodiment, the cultivation on lignocellulosic hydrolysate renders microorganism effective in reducing gastrointestinal permeability. In one embodiment, a composition for use of the invention enhances gastrointestinal health by reducing gastrointestinal permeability.

Provided herein are means to improve gastrointestinal health through a reduction in intestinal permeability in humans and animals. In particular, the means improve the gastrointestinal health of mammals such as humans, pigs, dogs, and cats; poultry such as chickens, turkeys, and ducks; fish such as salmon, trout, bass, bream, cod, catfish, and tilapia; invertebrates such as a shrimp, lobster, crab, octopus, squid, oyster, clam, or mussel. In one embodiment, a composition for use of the invention enhances gastrointestinal health of a human patient or animal patient by reducing gastrointestinal permeability. In one embodiment, a composition of the invention can be used to enhance livestock farming by enhancing gastrointestinal health of livestock, preferably by reducing gastrointestinal permeability of said livestock. In one embodiment, a composition of the invention can be used to enhance gastrointestinal health of a domestic animal such as a pet, preferably by reducing gastrointestinal permeability of said domestic animal.

In the context of this invention, improving gastrointestinal health relates to reducing gastrointestinal permeability and/or alleviating other pathological conditions, e.g. reducing inflammation or the inflammatory cascade, or modulating the microbiota towards favorable microorganisms, either through stimulating proliferation of beneficial microorganisms or reducing harmful microorganisms. In a preferred embodiment, gastrointestinal health of a patient, e.g. animal or human, is increased by reducing gastrointestinal permeability of said patient. Increased gastrointestinal permeability is associated with increases in inflammation or the inflammatory cascade, modulation of the microbiota towards harmful microorganisms either through proliferation or reduction to beneficial microorganisms, and is associated with the development of diseases such as Crohn's disease, irritable bowel syndrome, celiac disease, and infectious diarrhea. Gastrointestinal health may further be manifested as improvements to conditions of necrotic enteritis, diarrhea/scours, gastrointestinal discomfort, or general morbidity. In a preferred embodiment, a gastrointestinal health is increased by reducing gastrointestinal permeability.

In one embodiment, microorganisms obtain the capacity to reduce gastrointestinal permeability by cultivation of said microorganisms on lignocellulosic hydrolysate. Particularly, microorganisms cultivated on or with lignocellulosic hydrolysate are effective in reducing gastrointestinal permeability. In contrast thereto, microorganisms not cultivated on or with lignocellulosic hydrolysate, e.g. microorganisms cultivated on dextrose, are not effective in reducing gastrointestinal permeability. In one embodiment, cultivation of microorganism on or with lignocellulosic hydrolysate provides said microorganisms a gastrointestinal permeability-reducing capacity. In one embodiment, a composition of the invention comprises microorganisms cultivated on or with lignocellulosic hydrolysate capable of reducing gastrointestinal permeability. In one embodiment, a composition of the invention is capable of reducing gastrointestinal permeability, preferably by means of microorganisms comprised in said composition.

The lignocellulosic hydrolysate used to cultivate microorganisms is derived and/or prepared from lignocellulosic material and/or biomass. In one embodiment, the terms “derive from” and “prepare from”, or “derived from” and “prepared from”, are used interchangeably. The lignocellulosic material, preferably lignocellulosic hydrolysate, described herein is preferably derived from biomass, e.g. by chemical, enzymatic, physicochemical, or physical breakdown, or combinations thereof, of lignocellulosic material into its component parts. The term “lignocellulosic hydrolysates” refers to sugars and other break-down products prepared from lignocellulosic material and/or biomass. For example, lignocellulosic hydrolysates comprise C5 and C6 sugars (including monomers, dimers, and oligomers). In one embodiment, lignocellulosic hydrolysate further comprises an organic compound such as furfural and/or an inorganic compound such as ash. Hydrolysates typically comprise carbohydrates (e.g. C6 and/or C5 sugars), degradation products (e.g. from sugars and lignin), and further minor components. In one embodiment, the term “carbohydrate-rich” relates to lignocellulosic hydrolysate having a high amount of carbohydrates, e.g. C6 and/or C5 sugars. In one embodiment, said amount of carbohydrates relates to a weight percentage (g of dry carbohydrate/g of dry matrix). In one embodiment, a “carbohydrate-rich” hydrolysate has an amount of carbohydrates of interest, e.g. C6 and/or C5 sugars, which is at least ≥80% by weight compared to the other components present in the hydrolysate. In one embodiment, a “carbohydrate-rich” hydrolysate has an amount of carbohydrates, e.g. C6 and/or C5 sugars, of at least ≥80% by weight of the hydrolysate, preferably ≥80% by weight of the hydrolysate dry weight. In one embodiment, a method for preparing lignocellulosic hydrolysate is a method known to a person skilled in the art, such as a method of preparing lignocellulosic hydrolysate using enzymes, acids, and/or physical treatment such as steam explosion, e.g. a method as described in [5]. In one embodiment, a treatment involved in preparing lignocellulosic hydrolysate, such as a physical, chemical, physicochemical, and/or biological treatment, is a pretreatment. In one embodiment, the composition of the invention comprises microorganisms cultivated on or with lignocellulosic hydrolysate, and further comprises lignocellulosic hydrolysate. In one embodiment, the composition comprising lignocellulosic hydrolysate comprises hydrolysis products and/or residues of lignocellulose. In one embodiment, lignocellulosic hydrolysate comprises hydrolysis products and/or residues of lignocellulose. Without wishing to be bound by any theory, the inventors believe that a presence of hydrolysis products and/or residues of lignocellulose during the growth of the microorganisms facilitates the resulting improvement in gastrointestinal permeability.

In one embodiment, the composition of the invention and/or the lignocellulosic hydrolysate comprise(s) any of monosaccharides, e.g. five carbon sugar(s) such as glucose and six carbons sugar(s) such as xylose; disaccharides, e.g. cellobiose; oligosaccharides; furfural; hydroxymethylfurfural; acetic acid, levulinic; formic acid; extractives, e.g. tannins, phenolics, tetraterpenes, alkaloids, fats, and/or waxes; lignin; lignin degradation products, e.g., sinapyl alcohol and guaiacol; and any combination thereof. In one embodiment, the composition of the invention and/or the lignocellulosic hydrolysate comprise(s) any of
monosaccharide six carbons sugar(s) (e.g., glucose) in an amount in the range of 0-300 g/L;
monosaccharide five carbon sugar(s) (e.g., xylose) in an amount in the range of 0-300 g/L;
disaccharides (e.g., cellobiose) in an amount in the range of 0-300 g/L;
oligosaccharides in an amount in the range of 0-300 g/L;
hydrolysis or degradation products
   e.g., furfural, hydroxymethylfurfural in an amount in the range of 0-200 g/L,
   e.g., acetic acid, levulinic, formic acid in an amount in the range of 0-200 g/L;
extractives (e.g., tannins, phenolics, tetraterpenes, alkaloids, fats/waxes) in an amount in the range of 0-200 g/L;
lignin in an amount in the range of 0-200 g/L;
lignin degradation products (e.g., sinapyl alcohol, guaiacol) in an amount in the range of 0-200 g/L; and any combination thereof.

In one embodiment, the composition of the invention and/or the lignocellulosic hydrolysate comprise(s) any of monosaccharide six carbons sugar(s) (e.g., glucose) in an amount in the range of 0-30 wt %; monosaccharide five carbon sugar(s) (e.g., xylose) in an amount in the range of 0-30 wt %; disaccharides (e.g., cellobiose) in an amount in the range of 0-30 wt %; oligosaccharides in an amount in the range of 0-30 wt %; furfural and/or hydroxymethylfurfural in an amount in the range of 0-20 wt %; acetic acid, levulinic, and/or formic acid in an amount in the range of 0-20 wt %; tannins, phenolics, tetraterpenes, alkaloids, fats, and/or waxes in an amount in the range of 0-20 wt %; lignin in an amount in the range of 0-20 wt %; lignin degradation products, e.g., sinapyl alcohol and/or guaiacol, in an amount in the range of 0-20 wt %; and any combination thereof.

In one embodiment, a biomass used to prepare a lignocellulosic hydrolysate comprises lignocellulosic material comprising 30 to 55 percent cellulose by weight and hemicellulose at about 15 to 40 percent by weight. In one embodiment, a biomass used to prepare a lignocellulosic hydrolysate comprises lignocellulosic material comprising at least one of (a) a wood material and/or (b) saw dust residue (e.g. an agricultural residue); wherein, preferably, the wood material comprises at least one of saw dust, saw dust residue, e.g. hard wood sawdust comprising oak wood residue from a wood processing facility, oak wood, maple wood, hickory wood, poplar wood, or other indigenous hard woods to North America. In one embodiment, agricultural residue comprises at least one of corn stover, wheat straw, oat straw, rice straw, sugar cane bagasse, sugar beet bagasse, empty fruit bunches from palm oil extraction, fermentation spent grains, other agricultural residue. In one embodiment, a biomass used to prepare a lignocellulosic hydrolysate comprises lignocellulosic material comprising wood residue and cellulose at about 35 to 45 percent cellulose by weight and hemicellulose at about 17 to 24 percent by weight.

In one embodiment, the biomass used to prepare and/or derive a lignocellulosic hydrolysate is a lignocellulosic biomass. In one embodiment, the term “biomass” refers to a lignocellulosic biomass. In one embodiment, the terms “biomass” and “lignocellulosic biomass” are used interchangeably. As used herein, the term “lignocellulosic biomass” is meant to refer to a biomass which contains, inter alia, cellulose, hemicellulose, lignin, water extractives, ethanol extractives, acetic acid, phenolic compounds, free sugars (e.g. glucose and sucrose), inorganic minerals, resins, rosins, tannins, etc. The “biomass” or “lignocellulosic biomass” may be derived from different materials, for example hardwood, softwood, paper, in particular recycled paper, waste paper, wood shavings, sawdust, forest trimmings, pulp, corn stover, corn cobs, corn fiber, straw, in particular wheat straw, barley straw, rice straw, sugar cane bagasse, switch grass, empty fruit bunches from palm oil trees/residues, forestry thinnings, any sort of native or non-native grass, energy crops, agricultural residues, manure from animal feeding operations, human waste, construction debris, or combinations thereof. In one embodiment, a lignocellulosic hydrolysate is a lignocellulosic hydrolysate as described in US 20170159076 A1.

The term “biomass fractionation” refers to a process of generating lignocellulosic hydrolysates from biomass and/or lignocellulosic material. The source of lignocellulosic material can be, for example, hardwood, but also includes softwood and other materials of vegetal origin such as bagasse, stover, and related material. For example, biomass and/or lignocellulosic material can be any of forestry products and residues, e.g. wood and sawdust, agricultural crops, agricultural products, agricultural residues, e.g. corn, sugar cane, bagasse, and stover, crops e.g. energy crops, and waste products, e.g. food waste, solid paper waste, and spent liquors. Methods for production of lignocellulosic hydrolysates are known in the art. In one embodiment, biomass fractionation is a fractionation of biomass into its basic chemical components lignin, sugars, and/or fibers. In one embodiment, when referring to lignocellulosic hydrolysate being prepared via biomass fractionation followed by an enzymatic hydrolysis, the term “one or more of a fractionated biomass” relates to one or more parts, e.g. fractions, of a fractionated biomass and/or to one or more fractionated biomasses. In one embodiment, the term “enzymatic hydrolysis of a fractionated biomass” comprises enzymatic hydrolysis of one or more parts, e.g. fractions, of a fractionated biomass and/or to enzymatic hydrolysis of one or more fractionated biomasses. In one embodiment, biomass fractionation comprises any of thermochemical treatment; steam explosion; hot water extraction; autohydrolysis; sub critical water treatment; super critical water treatment; acid treatment, e.g. strong acid treatment or mild acid treatment; alkaline treatment, e.g. lime treatment or ammonia treatment; organic solvent treatment, e.g. treatment using alcohol(s) or organic acid(s); mechanical treatment; thermomechanical treatment; ionic liquid treatment; enzymatic treatment; and any combination thereof. In one embodiment, providing said lignocellulosic hydrolysate comprises treating biomass, particularly lignocellulosic biomass, using any of thermochemical treatment; steam explosion; hot water extraction; autohydrolysis; sub critical water treatment; super critical water treatment; acid treatment, e.g. strong acid treatment or mild acid treatment; alkaline treatment, e.g. lime treatment or ammonia treatment; organic solvent treatment, e.g. treatment using alcohol(s) or organic acid(s); mechanical treatment; thermomechanical treatment; ionic liquid treatment; enzymatic treatment; and any combination thereof. In one embodiment, the composition of the invention comprises microorganisms cultivated on or with lignocellulosic hydrolysate, wherein said lignocellulosic hydrolysate is obtained by subjecting lignocellulosic biomass to any of thermochemical treatment; steam explosion; hot water extraction; autohydrolysis; sub critical water treatment; super critical water treatment; acid treatment, e.g. strong acid treatment or mild acid treatment; alkaline treatment, e.g. lime treatment or ammonia treatment; organic solvent treatment, e.g. treatment using alcohol(s) or organic acid(s); mechanical treatment; thermomechanical treatment; ionic liquid treatment; enzymatic treatment; and any combination thereof.

In one embodiment, the lignocellulosic hydrolysate is obtained by subjecting lignocellulosic biomass to a) acid hydrolysis, e.g. dilute acid hydrolysis; b) acid hydrolysis, e.g. dilute acid hydrolysis, followed by steam-explosion and enzymatic hydrolysis; c) hemicellulose hydrolysis, followed by super-critical enzymatic hydrolysis; d) acid hydrolysis, e.g. dilute acid hydrolysis, then a second stage strong acid treatment and enzymatic hydrolysis; e) autohydrolysis, single stage steam-explosion pretreatment and enzymatic hydrolysis; f) autohydrolysis without acid addition, single state steam-explosion pretreatment and enzymatic hydrolysis; g) hemicellulose hydrolysis, followed by super-critical enzymatic hydrolysis, and dilute acid hydrolysis followed by steam-explosion and enzymatic hydrolysis; h) acid hydrolysis, e.g. extruder-based dilute acid hydrolysis, and enzymatic hydrolysis; and any combination thereof.

The term “microorganism” refers to microorganisms known to a person skilled in the art such as yeasts, fungi, bacteria, and microalgae, in particular to torula yeast and microorganisms other than torula yeast including, but not limited to, other yeasts, fungi, bacteria, and microalgae. In particular, but not limited to, yeasts of the genus Candida, Saccharomyces, Kluyveromyces, and Cyberlindnera, fungi of the genus Aspergillus, Paecilomyces, Pleurotus, and Trichoderma, bacteria of the genus Bacillus and Lactobacillus, and microalgae of the genus Spirulina and Chlorella. Microorganisms not cultivated on lignocellulosic hydrolysates show lower efficacy compared to microorganisms cultivated on lignocellulosic hydrolysate. In one embodiment, the composition of the invention comprises microorganisms cultivated on or with lignocellulosic hydrolysate, wherein the microorganisms comprise yeast, preferably yeast selected from Cyberlindnera sp., more preferably torula yeast, and/or derivatives, fractions, hydrolysates and/or extracts of such yeast, e.g. yeast hydrolysates and yeast extracts.

In one embodiment, when referring to “microorganisms”, said term also comprises derivatives, fractions, hydrolysates and extracts of such microorganisms, such as yeast-derived additives, yeast fractions, yeast hydrolysates and/or yeast extracts. In one embodiment, after said cultivation of said microorganisms, said microorganisms are further processed by derivation, fractionation, hydrolysis, and/or extraction, preferably prior to using said microorganisms in a composition. In one embodiment, after said cultivation of said microorganisms, said microorganisms are further processed by derivation, fractionation, hydrolysis, and/or extraction, and said processed microorganism derivatives, microorganism derivatives, microorganism hydrolysates, and/or microorganism extracts are used in a composition and/or in a method of preventing and/or treating a gastrointestinal order. In one embodiment, after said cultivation of said microorganisms, said microorganisms are further processed by derivation, fractionation, hydrolysis, and/or extraction prior to using said microorganisms in a composition and/or method of the invention, and microorganism derivatives, microorganism derivatives, microorganism hydrolysates, and/or microorganism extracts obtained from such further processing are used in a composition of the invention, in a method of preparing a composition, in a method of preventing or treating of the invention, in a method of reducing gastrointestinal permeability of the invention, and/or in a method of enhancing gastrointestinal health of the invention.

In one embodiment, derivatives, fractions, hydrolysates and/or extracts of microorganisms are prepared using enzymes, thermal treatment, and/or mechanical treatment. In one embodiment, when preparing hydrolysates, all fractions remain in the final product. In one embodiment, when preparing extracts, a portion of the microorganism extract is removed by physical (e.g. centrifugation) and/or chemical means (e.g. acid precipitation), preferably a carbohydrate fraction is removed. In one embodiment, derivatives, fractions, hydrolysates and/or extracts of microorganisms cultivated on lignocellulosic hydrolysate are capable of enhancing gastrointestinal health, preferably by reducing gastrointestinal permeability. In one embodiment, derivatives, fractions, hydrolysates and/or extracts of microorganisms are capable of enhancing gastrointestinal health, preferably by reducing gastrointestinal permeability, if said microorganisms further processed to obtain derivatives, fractions, hydrolysates and/or extracts thereof are microorganisms cultivated on lignocellulosic hydrolysate. In one embodiment, derivatives, fractions, hydrolysates and/or extracts of microorganisms cultivated on lignocellulosic hydrolysate are capable of enhancing gastrointestinal health by reducing gastrointestinal permeability, in contrast to derivatives, fractions, hydrolysates and/or extracts of microorganisms not cultivated on lignocellulosic hydrolysate which are not capable of enhancing gastrointestinal health by reducing gastrointestinal permeability. In one embodiment, cultivation of microorganisms on lignocellulosic hydrolysate surprisingly provides them with a capacity to reduce gastrointestinal permeability. In one embodiment, a composition of the invention is highly advantageous in that it allows to reduce gastrointestinal permeability.

In one embodiment, a microorganism is Torula yeast. Torula yeast refers to the yeast whose scientific name is Cyberlindnera jadinii, but is also referred to by its synonym Candida utilis. In one embodiment, a composition comprises primary grown torula yeast cultivated on or with an appropriate medium containing lignocellulosic hydrolysates. In one embodiment, the term “cultivated on or with” means cultivation using at least a particular medium and/or substrate, i.e. cultivation “on” said medium and/or substrate, and/or cultivation “with” said medium and/or substrate. In one embodiment, whether microorganism are cultivated “on” or “with” said medium and/or substrate, particularly “on” or “with” said lignocellulosic hydrolysate, may depend on the type of cell culture used, e.g. whether an adherent culture or suspension culture is used. In one embodiment, the terms “cultivated on” and “cultivated with” are used interchangeably. In one embodiment, the terms “cultivated on or with”, “cultivated on” and “cultivated with” are meant to refer to “cultivated using”. In one embodiment, microorganisms having been subjected to a cultivation on or with lignocellulosic hydrolysate are microorganisms cultivated on or with lignocellulosic hydrolysate. In one embodiment, such cultivation on or with lignocellulosic hydrolysate is for a period of 0.1 to 600 h, preferably 1 to 350 h, and/or at a temperature of 15 to 60° C., preferably 20 to 45° C., for example as a batch, fed-batch, and/or continuous cultivation. In one embodiment, lignocellulosic hydrolysate is used as a medium, as a component of a medium, as a substrate, and/or as a component of a substrate. In one embodiment, the appropriate medium includes lignocellulosic hydrolysate(s). In one embodiment, when referring to microorganism cultivated on lignocellulosic hydrolysate, such microorganisms are cultivated on at least lignocellulosic hydrolysate, wherein said lignocellulosic hydrolysate may comprise further components such as supplements, and/or wherein said lignocellulosic hydrolysate may be comprised by any substance, e.g. comprised by a cell culture medium. In one embodiment, the cell biomass resulting from a cultivation of microorganism, preferably resulting from a cultivation of microorganisms using lignocellulosic hydrolysate, may be heat-inactivated and/or dried to a suitable moisture content, e.g. a moisture content of <10% by weight. In one embodiment, after said cultivation on or with lignocellulosic hydrolysate, said microorganisms are heat-inactivated and/or dried, prior to preparing a composition using said microorganisms. In one embodiment, a composition of the invention comprises heat-inactivated and/or dried microorganisms. The cell biomass resulting from a cultivation of microorganisms may also be subjected to further processing including, but not limited to, centrifugation, autolysis, derivation, fractionation, hydrolysis, extraction, and/or washing e.g. using one or more alcohols, acids, and/or solvents. In one embodiment, a method of preparing a composition comprises a step of preparing a derivative, fraction, hydrolysate and/or extract of said microorganisms, preferably using enzymes, thermal treatment, and/or mechanical treatment. In one embodiment, a method of preparing a composition of the invention comprises a step of centrifugation of said microorganisms, optionally a step of centrifugation of derivatives, fractions, hydrolysates and/or extracts of said microorganisms. In one embodiment, a method of preparing a composition of the invention comprises a step of autolysis of said microorganisms, optionally a step of autolysis of derivatives, fractions, hydrolysates and/or extracts of said microorganisms. In one embodiment, a method of preparing a composition of the invention comprises a step of derivation of said microorganisms, optionally a step of derivation of derivatives, fractions, hydrolysates and/or extracts of said microorganisms. In one embodiment, a method of preparing a composition of the invention comprises a step of fractionation of said microorganisms, optionally a step of fractionation of derivatives, fractions, hydrolysates and/or extracts of said microorganisms. In one embodiment, a method of preparing a composition of the invention comprises a step of hydrolysis of said microorganisms, optionally a step of hydrolysis of derivatives, fractions, hydrolysates and/or extracts of said microorganisms. In one embodiment, a method of preparing a composition of the invention comprises a step of extraction of said microorganisms, optionally a step of extraction of derivatives, fractions, hydrolysates and/or extracts of said microorganisms. In one embodiment, a method of preparing a composition of the invention comprises a step of washing of said microorganisms, optionally a step of washing of derivatives, fractions, hydrolysates and/or extracts of said microorganisms, e.g. washing using one or more alcohols, acids, and/or solvents. In one embodiment, when referring to “microorganisms”, said term also comprises derivatives, fractions, hydrolysates and/or extracts of said microorganisms.

Methods for the production, preparation, and cultivation of microorganisms such as yeast using standard substrates and/or cell media are known in the art. However, microorganism cultivated on or with lignocellulosic hydrolysate as a substrate and/or medium surprisingly exhibit gastrointestinal permeability-reducing effects. The inventors have surprisingly detected that microorganisms such as yeast cultivated on or with lignocellulosic hydrolysate have the highly advantageous capacity to enhance gastrointestinal health by reducing gastrointestinal permeability. Furthermore, the inventors have surprisingly detected that microorganisms cultivated on or with lignocellulosic hydrolysate are highly effective in reducing gastrointestinal permeability, and that such microorganisms cultivated on or with lignocellulosic hydrolysate show statistically significant higher efficacy compared to microorganisms not cultivated on or with lignocellulosic hydrolysates. Vice versa, the inventors have detected that microorganisms such as yeast not cultivated on lignocellulosic hydrolysates show statistically lower efficacy compared to microorganisms such as yeasts cultivated on lignocellulosic hydrolysate.

When referring to a “microorganism”, such term is also meant to refer to derivatives of such microorganism, such as extracts and hydrolysates of such organism. In one embodiment, a derivative is a biologically active derivative, and such biologically active derivative also achieves the effect of the present invention. For example, when referring to “torula” and/or “torula yeast”, such term is also meant to refer to derivatives of torula yeast including, but not limited to, yeast extracts and yeast hydrolysates. Methods for the production of microorganisms and derivatives thereof, e.g. yeast and yeast derivatives, are known in the art.

In one embodiment, a method of preparing a composition comprises culturing microorganism using lignocellulosic hydrolysate as a medium and/or substrate, optionally further comprising supplementing said medium and/or substrate, preferably with any supplement selected from cell culture medium, nutrients, vitamins, salt such as mineral salt, water, buffer e.g. pH buffer, a nitrogen source, a source of amino acids e.g. amino acids or yeast extract, and a carbon source, more preferably any of nutrients, vitamins, and salt such as mineral salt. In one embodiment, the term “cultivated on or with lignocellulosic hydrolysate”, as used herein, relates to growing microorganisms using lignocellulosic hydrolysate as a substrate and/or medium, optionally further comprising supplements. In one embodiment, said using lignocellulosic hydrolysate as a substrate and/or medium further comprises adding one or more supplements, substrates and/or media to such lignocellulosic hydrolysate, e.g. a supplement selected from cell culture medium, nutrients, vitamins, salt such as mineral salt, water, buffer e.g. pH buffer, a nitrogen source, a source of amino acids e.g. amino acids or yeast extract, and a carbon source, more preferably any of nutrients, vitamins, and salt such as mineral salt. In one embodiment, cultivated on or with lignocellulosic hydrolysate relates to cultivated on or with at least lignocellulosic hydrolysate, wherein said at least lignocellulosic hydrolysate can be supplemented with any supplement, substrate and/or media, and/or wherein said lignocellulosic hydrolysate can be comprised by, e.g. added to, any medium such as cell culture medium.

In one embodiment, culturing said microorganisms comprises adding a supplement to a medium. In one embodiment, culturing said microorganisms on or with lignocellulosic hydrolysate comprises adding a supplement to said lignocellulosic hydrolysate and/or mixing lignocellulosic hydrolysate with at least a cell culture medium which optionally comprises further supplements such as nutrients, vitamins, and/or mineral salts. In one embodiment, the term “nutrient”, as used herein, relates to any substance used by an organism to survive, grow, and reproduce. In one embodiment, a nutrient is any of vitamin A, vitamin C, vitamin D, vitamin K, α-tocopherol (vitamin E), thiamin (vitamin B₁), riboflavin (vitamin B₂), niacin (vitamin B₃), pantothenic acid (vitamin B₅), vitamin B₆, biotin (vitamin B₇), folate (vitamin B₉), cobalamin (vitamin B₁₂), choline, calcium, chloride, chromium, copper, fluoride, iodine, iron, magnesium, manganese, molybdenum, phosphorus, potassium, selenium, sodium, zinc, nitrogen, sulfur, oxygen, carbon, hydrogen, and cobalt. In one embodiment, the term “supplement”, as used herein in the context of cell cultures and/or cultivation, relates to a cell culture supplement and/or to any agent that stimulates, enhances and/or promotes cell growth, e.g. a nutrient.

In one embodiment, the terms “culturing said microorganisms” and “cultivation of said microorganisms” are used interchangeably. In one embodiment, culturing said microorganisms comprises a step of fermentation. In a preferred embodiment, a method of preparing a composition of the invention comprises drying said microorganisms and/or mixture, preferably after said culturing, more preferably after said culturing and prior to said obtaining. The term “fermentation”, as used herein, relates to a metabolic process that produces chemical changes in organic substrates through the action of enzymes, e.g. the extraction of energy from carbohydrates in the absence of oxygen and/or a process in which the activity of microorganisms brings about a desirable change to a material such as a foodstuff or beverage. In one embodiment, fermentation is a primary means of microorganisms for producing adenosine triphosphate (ATP) by the degradation of organic nutrients anaerobically. In one embodiment, a “step of fermentation” comprises culturing said microorganisms anaerobically. Typically, a step of fermentation can be performed using a batch process, fed-batch process, and/or continuous fermentation. In a batch process, the ingredients are combined and the reactions proceed without any further input. Fed-batch fermentation is a variation of batch fermentation in which some of the ingredients, e.g. nutrients, are added during the fermentation. In continuous fermentation, ingredients, e.g. substrates, are added and final products removed continuously.

In one embodiment, a composition of the invention comprises at least microorganisms cultivated on or with lignocellulosic hydrolysate, and optionally comprises further components such as lignocellulosic hydrolysate, a carrier, and/or food ingredients. In one embodiment, a composition is formulated as a food product, food formulation or feed formulation. The composition of the invention is highly advantageous in that it effectively enhances gastrointestinal health by reducing gastrointestinal permeability. In one embodiment, the composition of the invention contains a nucleic acid content of less than 10% by weight, preferably <3% by weight, e.g. <1% by weight. The nucleic acid content may be achieved through biological means or through mechanical means, such as enzymatic digestions using a DNAse and/or RNAse, by heat-treatment, and/or by precipitation. The composition of the invention optionally comprises further suitable carriers for microorganisms cultivated on lignocellulosic hydrolysates such as starches e.g. corn starch, dextrins, oligosaccharides, fibers e.g. cellulose, proteins e.g. soya, and oils e.g. vegetable oil, or any suitable ingredients e.g. food ingredients or feed ingredients. In one embodiment, the composition is substantially free of contaminants such as heavy metals and mycotoxins. In one embodiment, a carrier is an inactive ingredient that does not have an effect, particularly therapeutic effect, on the microorganisms and/or the patient. In one embodiment, a carrier is any of starches e.g. corn starch, dextrins, oligosaccharides, fibers e.g. cellulose, proteins e.g. soya, oils e.g. vegetable oil, complex feed, compound feed, food, and prepared food.

In one embodiment, a composition for use in the treatment of a gastrointestinal disorder is administered to a patient in need thereof, e.g. daily and/or orally. In one embodiment, a composition for use is administered joints with food intake, for example by means of a food formulation or feed formulation comprising said composition. In one embodiment, a food or feed is admixed with said composition. In one embodiment, a composition, e.g. a feed composition or food composition, comprising torula yeast and/or other microorganisms cultivated on lignocellulosic hydrolysates, is orally administered, preferably daily. In one embodiment, such composition is orally administered in a concentration of 0.001-20% by weight of a feed formulation, food formulation, or a daily food consumption, preferably 0.1-20% by weight of a feed formulation, food formulation, or a daily food consumption, e.g. 0.1-10% by weight, preferably daily. The appropriate proportions are determined by someone skilled in the art of feed formulation or food formulation, e.g. nutritionist. In one embodiment, the composition is administered daily and/or with every meal or feed. In one embodiment, the composition is formulated in any suitable form to be administered to a human or animal patient, e.g. in the form of a powder, granules, a tablet, a pill, a capsule, a dispersion, a solution, a suspension, an emulsion, a liquid, a liquid concentrate, an oil mixture, a bar, a stick, a food product, a food supplement, a drink, a food formulation, a feed formulation, or a lozenge, optionally further comprising food ingredients or beverage ingredients. In one embodiment, the terms “food product”, “food supplement”, “food formulation” and “feed formulation” are used interchangeably. In one embodiment, the term “food product” relates to any product having a nutritional value, e.g. a food product formulated as food formulation or feed formulation, such as animal food or animal feed. In one embodiment, a food product or food formulation is a prepared food or beverage. In one embodiment, a food product or feed formulation is a complex feed, a compound feed, or beverage.

In one embodiment, if a composition is formulated in a form of a powder, granules, a tablet, a pill, a capsule, a dispersion, a solution, a suspension, an emulsion, a liquid, a liquid concentrate, an oil mixture, a bar, a stick, a food product, a food supplement, a drink, a food formulation, a feed formulation, or a lozenge, such powder, granules, tablet, pill, capsule, dispersion, solution, suspension, emulsion, liquid, liquid concentrate, oil mixture, bar, stick, food product, food supplement, drink, food formulation, feed formulation, or lozenge comprise(s) said composition and optionally further ingredients, such as ingredients having nutritional value or flavor, e.g. food or beverage components. In one embodiment, a “beverage” is a liquid intended for human consumption or animal consumption. For example, a beverage is any of water, milk, juice, coffee, tea, hot chocolate, soft drinks, alcoholic beverages, non-alcoholic beverages, and milk replacer. In one embodiment, if intended for animal consumption, a beverage is, for example, water, milk, milk replacer.

In one embodiment, a “daily food consumption”, as used herein, relates to an amount of food consumed per day, e.g. animal food or food for humans. In one embodiment, a daily food consumption relates to an amount of feed per day. In a preferred embodiment, a composition of the invention is administered at a dosage in the range of from 0.001 to 50% by weight, preferably of from 0.1 to 20% by weight, of a total weight of a daily food consumption, i.e. 0.1 to 20% by weight of the total food or feed consumed by a patient per day is a composition of the invention; and/or in the range of 0.5-100 g/kg bodyweight per day, preferably 2-10 g/kg bodyweight per day, e.g. about 5.5 g/kg bodyweight per day, i.e. 0.5-100 g of the composition is administered for each kg bodyweight of said patient per day.

In one embodiment, a composition of the invention is advantageous in that microorganisms grown on lignocellulosic hydrolysates have an ability to ameliorate gastrointestinal permeability, i.e. reduce gastrointestinal permeability, whereas microorganisms grown on dextrose do not. The inventors have surprisingly detected that the gastrointestinal permeability is ameliorated by microorganisms and/or a compound produced by said microorganisms cultivated on lignocellulosic hydrolysate. The inventors have detected that such ameliorating activity is not caused by a carry over product from the lignocellulosic hydrolysate and instead is caused by the microorganism and/or a substance produced by the microorganism itself. The inventors have verified that the lignocellulosic hydrolysate by itself does not have a positive effect on permeability. The inventors have detected that, surprisingly, microorganisms grown on lignocellulosic hydrolysate reduce gastrointestinal permeability. In one embodiment, the terms “gastrointestinal permeability” and “intestinal permeability” are used interchangeably. The inventors have surprisingly found that microorganism cultivated on or with lignocellulosic hydrolysate effectively enhance gastrointestinal health by reducing gastrointestinal permeability, in contrast to lignocellulosic hydrolysate and in contrast to microorganism not cultivated on lignocellulosic hydrolysate.

The term “animal feed” and/or “animal food”, as used herein, relates to food given to domestic animals, e.g. livestock in the course of animal husbandry, e.g. in the form of a food product, food formulation, and/or feed formulation. In one embodiment, animal feed is any of bird feed, cat foot, cattle feed, dog food, equine food, fish feed, pet food, pig feed, poultry feed, and/or sheep feed. In one embodiment, a composition of the invention is administered as part of a compound feed and/or within a prepared food and/or with a carrier. In one embodiment, a food product, food formulation, and/or feed formulation of the invention is animal feed and/or animal food. In one embodiment, a composition of the invention is formulated for administration to an animal, preferably formulated as a compound feed, as a supplement, and/or as a formulation to be mixed with compound feed, e.g. a powder. In one embodiment, when referring to a “supplement” in the context of a composition, administration and/or formulation, such supplement is a food supplement and/or feed supplement. In one embodiment, a composition of the invention is formulated for administration to a human, preferably formulated as a formulation to be directly administered, e.g. a pill, powder, capsule, tablet, and/or formulated as a formulation to be mixed with food, e.g. prepared food, such as meat analogues, patties, spreads, dressings, baked goods, soups, bars, crisps, and/or crackers, and/or formulated as a food product, food supplement, or food formulation.

In one embodiment, food formulation or feed formulation comprises one or more plant proteins up to 70% by weight of the total weight of the formulation. In one embodiment, plant proteins are vegetable proteins. In one embodiment, exemplary plant proteins are selected from soybean meal, soy protein concentrate, soy protein isolate, wheat gluten, corn gluten, pea protein concentrate, pea protein isolate, and combinations thereof.

In one embodiment, a food formulation or feed formulation comprises one or more non-fiber plant carbohydrate sources up to 70% by weight of the formulation. In one embodiment, exemplary plant carbohydrate sources are selected from corn, dried distiller's grains, wheat, sorghum, barley, milo, and combinations thereof.

In one embodiment, a food formulation or feed formulation comprises one or more animal proteins up to 70% by weight of the formulation. In one embodiment, exemplary animal proteins are selected from fish meal, krill meal, poultry meal, poultry by-product meal, bovine plasma, bovine blood meal, porcine plasma, porcine blood meal, meat, bone meal, and combinations thereof.

In one embodiment, a food formulation or feed formulation comprises one or more animal proteins up to 70% by weight of the formulation, and further comprises a composition of the invention. In one embodiment, a food formulation or feed formulation comprises one or more animal proteins up to 70% by weight of the formulation, and further comprises one or more plant proteins and a composition of the invention. In one embodiment, a food formulation or feed formulation comprises one or more plant proteins up to 70% by weight of the formulation, and further comprises a composition of the invention. In one embodiment, said microorganisms comprised by said composition comprise or consist of torula yeast. In one embodiment, said composition comprises microorganisms cultivated on or with lignocellulosic hydrolysate, wherein said microorganisms comprise or consist of torula yeast.

In one embodiment, a composition of the invention is included in a food formulation or feed formulation at the expense of both animal protein and plant protein at appropriate proportions. The appropriate proportions are determined by someone skilled in the art of food formulations and feed formulations, e.g. a nutritionist. In one embodiment, a food formulation or feed formulation comprises a composition of the invention in an amount of 0.01-20% by weight of the total weight of the formulation. In a preferred embodiment, a food formulation or feed formulation is supplemented with a composition of the invention.

In one embodiment, a composition, food formulation, and/or feed formulation further comprises any of synthetic amino acids, vitamin-mineral premixes, supplemental starches, and supplemental fats. In one embodiment, a composition, food formulation, and/or feed formulation further comprises amino acids in an amount of 0-5% by weight of the formulation, preferably crystalline amino acids. In one embodiment, amino acids such as crystalline amino acids are amino acids such as, but not limited to, lysine, methionine, and taurine. In one embodiment, an amino acid is a free acid, analog, and/or salt form.

In one embodiment, a composition, food formulation, and/or feed formulation comprises 0-10% by weight of a calcium source such as, but not limited to, calcium carbonate. In one embodiment, a composition, food formulation, and/or feed formulation comprises 0-10% by weight of a phosphorus source such as, but not limited to, mono- or di-calcium phosphate. In one embodiment, a composition, food formulation, and/or feed formulation comprises 0-10% by weight of a sodium and chloride source such as, but not limited to, sodium chloride. In one embodiment, a composition, food formulation, and/or feed formulation comprises 0-30% by weight of a fat source such as, but not limited to, fish oil or poultry fat. In one embodiment, a composition, food formulation, and/or feed formulation comprises 0-3% by weight of vitamins and/or minerals, e.g. a vitamin-mineral premix. In one embodiment, a composition, food formulation, and/or feed formulation comprises 0-2% by weight of a supplemental choline source such as, but not limited to, choline chloride. In one embodiment, a vitamin-mineral premix comprises any combination of vitamins A, D, E, K, B₁, B₂, B₃, B₅, B₆, B₇, B₉, B₁₂, and C, and minerals chromium, copper, fluorine, iodine, iron, magnesium, manganese, molybdenum, potassium, selenium, and zinc. As used here, vitamin-mineral premixes refer to both organic and inorganic sources.

In one embodiment, a composition, food formulation, and/or feed formulation comprises one or more additional zootechnical additives such as, but not limited to, enzymes, palatants, essential oils, organic acids, antibiotics, ionophores, pigments, and anti-oxidants. In one embodiment, a composition, food formulation, and/or feed formulation comprises Zootechnical additives in an amount of 0.001-5% by weight. In one embodiment, said composition, food formulation, and/or feed formulation comprises a component selected from an excipient, a sweetener, a sugar, a starch, a preservative, a flavoring agent, a nutritional oil such as a vegetable oil or fish oil, water, a milk such as a cow's milk, a soya milk, or an almond milk, a juice e.g. fruit juice or vegetable juice, or a colorant.

In one embodiment, the gastrointestinal disorder is selected from increased gastrointestinal permeability, leaky gut syndrome, inflammatory bowel disease, Crohn's disease, ulcerative colitis, irritable bowel syndrome, necrotic enteritis, colibacillosis, diarrhea/scours, porcine epidemic diarrhea virus, soybean meal induced enteritis, transient soy hypersensitivity, celiac disease, post-weaning diarrhea, and necrotic enteritis; preferably selected from leaky gut syndrome, Crohn's disease, irritable bowel syndrome, diarrhea/scours, and celiac disease. In one embodiment, the gastrointestinal disorder, particularly the gastrointestinal disease, is associated with increased gastrointestinal permeability. In one embodiment, the gastrointestinal disorder, particularly the gastrointestinal disease, results from and/or relates to increased gastrointestinal permeability, such as a disorder selected from leaky gut syndrome, Crohn's disease, irritable bowel syndrome, diarrhea/scours, and celiac disease. In one embodiment, the composition of the invention is used in a method of preventing and/or treating a gastrointestinal disorder associated with increased gastrointestinal permeability, preferably a disorder selected from leaky gut syndrome, Crohn's disease, irritable bowel syndrome, diarrhea/scours, and celiac disease. In one embodiment, the composition of the invention reduces increased gastrointestinal permeability and thereby alleviates gastrointestinal disorders resulting from and/or relating to increased gastrointestinal permeability, such as leaky gut syndrome, Crohn's disease, irritable bowel syndrome, diarrhea/scours, and celiac disease. In a preferred embodiment, the gastrointestinal disorder is selected from leaky gut syndrome, Crohn's disease, irritable bowel syndrome, diarrhea/scours, and celiac disease. In one embodiment, in said method of preventing and/or treating a gastrointestinal disorder, the composition of the invention is administered to a patient in need thereof, wherein said patient is characterized by increased gastrointestinal permeability. In one embodiment, the term “increased gastrointestinal permeability” relates to a gastrointestinal permeability of a patient, e.g. human or animal, which is increased compared to the gastrointestinal permeability of a healthy individual, e.g. a healthy human or healthy animal. The composition for use of the invention is highly effective in alleviating a gastrointestinal disorder in a patient suffering from increased gastrointestinal permeability.

In one embodiment, a gastrointestinal disorder is any condition, disorder and/or disease involving increased gastrointestinal permeability, e.g. any of increased gastrointestinal permeability, leaky gut syndrome, inflammatory bowel disease, Crohn's disease, ulcerative colitis, irritable bowel syndrome, necrotic enteritis, colibacillosis, diarrhea/scours, porcine epidemic diarrhea virus, soybean meal induced enteritis, transient soy hypersensitivity, celiac disease, post-weaning diarrhea, necrotic enteritis, diabetes, rheumatoid arthritis, spondyloarthropathies, schizophrenia, cancer, fatty liver disease, atopy, and allergies. In one embodiment, a method of enhancing gastrointestinal health comprises preventing or treating a gastrointestinal disorder and/or reducing gastrointestinal permeability. In one embodiment, a method of reducing gastrointestinal permeability in a patient comprises preventing or treating a gastrointestinal disorder. In one embodiment, a method of preventing or treating a gastrointestinal disorder comprises enhancing gastrointestinal health and/or reducing gastrointestinal permeability in a patient. In one embodiment, an administration of a composition is an oral administration of said composition. In one embodiment, a “patient” is a human or an animal. In one embodiment, a composition for use is administered to a patient in need thereof. In one embodiment, the term “medium” relates to a cell culture medium. In one embodiment, the terms “food intake” and “food consumption” are used interchangeably. In one embodiment, the term “comprises” relates to “consists of”.

### BRIEF DESCRIPTION OF THE FIGURES

The present invention is now further described by reference to the following figures.

All methods mentioned in the figure descriptions below were carried out as described in detail in the examples.

FIG. 1 shows transepithelial electric resistance (TEER) across Caco-2 monolayer administered null (blank), torula yeast cultivated on dextrose (conventional), torula yeast cultivated on wood hydrolysate (woody), CM line (dotted red) represents disruption caused by PMA-activated THP-1 macrophages. 100% line represents initial TEER value. It is shown that administration of torula yeast cultivated on wood hydrolysates improves the TEER across the membrane to levels near those prior to membrane disruption in both pig and dog models (P<0.0001). Yeast cultivated on dextrose is not significantly different from the blank (P>0.05): indicating that gastrointestinal permeability remains. Accordingly, microorganisms cultivated on lignocellulosic hydrolysate effectively reduce gastrointestinal permeability, whereas microorganisms not cultivated on lignocellulosic hydrolysate do not effectively reduce gastrointestinal permeability.

FIG. 2 shows transepithelial electric resistance (TEER) across Caco-2 monolayer administered null (blank), torula yeast cultivated on dextrose (conventional), torula yeast cultivated on wood hydrolysate (woody), CM line (dotted red) represents disruption caused by PMA-activated THP-1 macrophages. 100% line represents initial TEER value. Accordingly, it is shown that administration of torula yeast cultivated on wood hydrolysates improves the TEER across the membrane in a pig model in a dose-dependent manner (P<0.0001). The lowest wood-based yeast concentration negatively impacted TEER values. Yeast cultivated on dextrose is not significantly different from the blank (P>0.05): indicating that gastrointestinal permeability remains. Accordingly, it is shown that a composition of the invention effectively reduces gastrointestinal permeability in a dose-dependent manner.

FIG. 3 shows transepithelial electric resistance (TEER) across Caco-2 monolayer administered null (blank), torula yeast cultivated on dextrose (conventional), torula yeast cultivated on wood hydrolysate (woody), CM line (dotted red) represents disruption caused by PMA-activated THP-1 macrophages. 100% line represents initial TEER value. Accordingly, it is shown that administration of torula yeast cultivated on wood hydrolysates as a pure product improves the TEER across the membrane to levels near those prior to membrane disruption (P<0.0001). Yeast cultivated on dextrose was not significantly different from the blank (P>0.05): indicating that gastrointestinal permeability remains.

FIG. 4 shows transepithelial electric resistance (TEER) across Caco-2 monolayer administered torula yeast cultivated on various wood hydrolysates obtained from various fractionation techniques: acid hydrolysis+steam explosion+enzymatic hydrolysis on hemicellulose stream (HH), super-critical water and enzymatic hydrolysis (RED), acid hydrolysis (TBR), acid-hydrolysis+STEAM-Ex+enzymatic hydrolysis on cellulose stream (EHH), auto-hydrolysis, steam explosion+enzymatic hydrolysis (BLU), auto-hydrolysis, steam explosion+enzymatic hydrolysis (WHI), Combination of RED+EHH (REDEH), acid hydrolysis (GRN). The positive control is sodium butyrate (NaB)-treated cells. CM line (dotted red) represents disruption caused by PMA-activated THP-1 macrophages. 100% line represents initial TEER value. It is shown that administration of torula yeast cultivated on different wood hydrolysates improves the TEER across the membrane to levels near those prior to membrane disruption (P<0.0001). Accordingly, microorganisms cultivated on lignocellulosic hydrolysate effectively reduce gastrointestinal permeability, whereas microorganisms not cultivated on lignocellulosic hydrolysate do not effectively reduce gastrointestinal permeability.

In the following, reference is made to the examples, which are given to illustrate, not to limit the present invention.

### EXAMPLES

### Example 1: Administration of Microorganisms Cultivated on Lignocellulosic Hydrolysates Improves the Transepithelial Electrical Resistance Across the Membrane

Dried, inactive torula yeast was cultivated on either wood-derived hydrolysates (Torula—woody) or on dextrose (Torula—conventional) in a 1,000 L bioreactor. Yeast was separated from the media in which it was cultivated, heat-inactivated, and dried to a moisture content of <10%.

Torula yeast was subjected to digestion that mimicked passage through the upper gastrointestinal tract (GIT) in a pig and dog, respectively. First, an oral phase containing amylase was applied to mimic mastication. The oral incubation was immediately followed by a gastric phase containing pepsin and salts (NaCl and KCl) to mimic digestion in the stomach. Following the gastric phase, and intestinal phase was applied containing trypsin, chymotrypsin, lipase, and α-amylase to mimic digestion in the small intestine. Free amino acids were removed from the digest using a cellulose dialysis membrane to mimic amino acid absorption. The exact conditions varied between the pig and dog models and reflected in vivo conditions in the respective species.

The Caco-2 cell line is a human intestinal epithelial-like cell line with an extensive history of use as a model in mammalian gastrointestinal research. It is the most widely used cell line for developing human GIT in vitro models. Its use has been approved by the United States Food & Drug Administration as a surrogate for human absorption/permeability testing (FDA, 2017). Caco-2 cells may be grown to a confluent monolayer that provides a physical and biochemical barrier to the passage of ions and small molecules. At this stage, cells are differentiated and polarized in such way that, both morphologically and functionally, they resemble the enterocyte lining in the human small intestine. When these cells are co-cultured with PMA-activated THP-1 macrophages (i.e., a human monocytic cell line), THP-1-induced inflammation occurs and causes the disruption of the Caco-2 monolayer.

The Caco-2 co-culture experiment was performed as described in [4]. Briefly, Caco-2 cells (HTB-37; American Type Culture Collection) were seeded in 24-well semi-permeable inserts. Caco-2 monolayers were cultured for 14 days, with three medium changes/week, until a functional cell monolayer with a transepithelial electrical resistance (TEER) was obtained. Cells were maintained in Dulbecco's Modified Eagle Medium (DMEM) containing glucose and glutamine and supplemented with HEPES and 20% (v/v) heat-inactivated (HI) fetal bovine serum (FBS).

THP1-Blue™ (InvivoGen) cells were maintained in Roswell Park Memorial Institute (RPMI) 1640 medium containing glucose and glutamine, supplemented with HEPES, sodium pyruvate and 10% (v/v) HI-FBS. THP1-Blue™ are THP1 human monocytes stably transfected with a reporter construct expressing a secreted alkaline phosphatase (SEAP) gene under the control of a promoter inducible by the transcription factor nuclear factor kappa B (NF-κB). Upon TLR activation (e.g. by lipopolysaccharide (LPS); isolated from Gram-negative bacteria), NF-κB becomes activated and induces the expression and secretion of SEAP. SEAP activity can then be measured in the supernatants by using the QUANTI-Blue reagent (InvivoGen). THP1-Blue™ cells were seeded in 24-well plates and treated with PMA that induces the differentiation of the cells into macrophage-like cells, which are able to adhere and are primed for TLR signaling.

Before setting up the co-culture, the transepithelial electrical resistance (TEER) of the Caco-2 monolayers was measured (=oh time point). Transepithelial electrical resistance is the measurement of electrical resistance across a cellular monolayer and is a very sensitive and reliable method to confirm the integrity and permeability of the monolayer. The TEER of an empty insert was subtracted from all readings to account for the residual electrical resistance of an insert. Then, the Caco-2-bearing inserts were placed on top of the PMA-differentiated THP1-Blue™ cells. Briefly, the apical compartment (containing the Caco-2 cells) was filled with upper GIT suspensions diluted to 60% in cell culture medium. Upper GIT suspensions were centrifuged (5000 rpm; 5′), but not filter-sterilized. Cells were also treated apically with Sodium butyrate (NaB) (Sigma-Aldrich) as positive control. The basolateral compartment (containing the THP1-Blue™ cells) was filled with Caco-2 complete medium. Cells were also exposed to Caco-2 complete medium in both chambers as control. Cells were treated for 24 h, after which the TEER was measured (=24 h time point). After subtracting the TEER of the empty insert, all 24 h values were normalized to its own oh value (to account for the differences in initial TEER of the different inserts) and are presented as percentage of initial value.

TEER data were analyzed with a one-way analysis of variance (ANOVA) with Dunnett's multiple comparisons test. (*) represents statistically significant differences between the CM control and the treatments. (*), (**), (***) and (****) represent p<0.05, p<0.01, p<0.001 and p<0.0001, respectively.

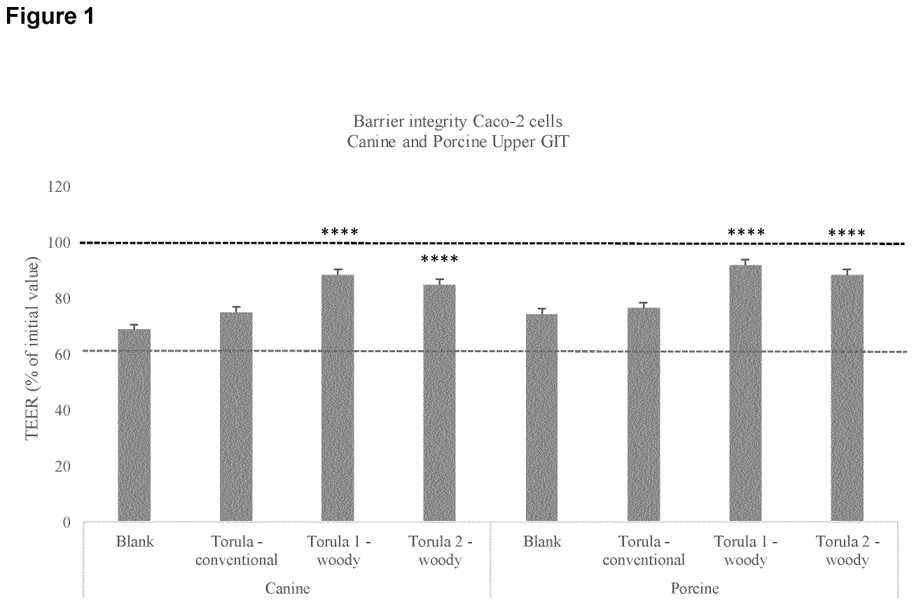

The CM line on FIG. 1 indicates the level of membrane disruption cause by PMA-activated THP-1 macrophages. Results of the experiment indicate that administration of torula yeast cultivated on wood hydrolysates improved the TEER across the membrane to levels near those prior to membrane disruption in both pig and dog models (P<0.0001). Yeast cultivated on dextrose was not significantly different from the blank (P>0.05): indicating that gastrointestinal permeability remained.

### Example 2: Administration of Microorganisms Cultivated on Lignocellulosic Hydrolysates Improves the Transepithelial Electrical Resistance in a Dose-Dependent Manner

Dried, inactive torula yeast was cultivated on either wood-derived hydrolysates (Torula—woody) or on dextrose (Torula—conventional) in a 1,000 L bioreactor. Yeast was separated from the media in which it was cultivated, heat-inactivated, and dried to a moisture content of <10%.

Torula yeast was subjected to digestion that mimicked passage through the upper gastrointestinal tract (GIT) in a pig and dog, respectively. First, an oral phase containing amylase was applied to mimic mastication. The oral incubation was immediately followed by a gastric phase containing pepsin and salts (NaCl and KCl) to mimic digestion in the stomach. Following the gastric phase, and intestinal phase was applied containing trypsin, chymotrypsin, lipase, and α-amylase to mimic digestion in the small intestine. Free amino acids were removed from the digest using a cellulose dialysis membrane to mimic amino acid absorption. The exact conditions varied between the pig and dog models and reflected in vivo conditions in the respective species.

The Caco-2 cell line is a human intestinal epithelial-like cell line with an extensive history of use as a model in mammalian gastrointestinal research. It is the most widely used cell line for developing human GIT in vitro models. Its use has been approved by the United States Food & Drug Administration as a surrogate for human absorption/permeability testing (FDA, 2017). Caco-2 cells may be grown to a confluent monolayer that provides a physical and biochemical barrier to the passage of ions and small molecules. At this stage, cells are differentiated and polarized in such way that, both morphologically and functionally, they resemble the enterocyte lining in the human small intestine. When these cells are co-cultured with PMA-activated THP-1 macrophages (i.e., a human monocytic cell line), THP-1-induced inflammation occurs and causes the disruption of the Caco-2 monolayer.

The Caco-2 co-culture experiment was performed as described in [4]. Briefly, Caco-2 cells (HTB-37; American Type Culture Collection) were seeded in 24-well semi-permeable inserts. Caco-2 monolayers were cultured for 14 days, with three medium changes/week, until a functional cell monolayer with a transepithelial electrical resistance (TEER) was obtained. Cells were maintained in Dulbecco's Modified Eagle Medium (DMEM) containing glucose and glutamine and supplemented with HEPES and 20% (v/v) heat-inactivated (HI) fetal bovine serum (FBS).

THP1-Blue™ (InvivoGen) cells were maintained in Roswell Park Memorial Institute (RPMI) 1640 medium containing glucose and glutamine, supplemented with HEPES, sodium pyruvate and 10% (v/v) HI-FBS. THP1-Blue™ are THP1 human monocytes stably transfected with a reporter construct expressing a secreted alkaline phosphatase (SEAP) gene under the control of a promoter inducible by the transcription factor nuclear factor kappa B (NF-κB). Upon TLR activation (e.g. by lipopolysaccharide (LPS); isolated from Gram-negative bacteria), NF-κB becomes activated and induces the expression and secretion of SEAP. SEAP activity can then be measured in the supernatants by using the QUANTI-Blue reagent (InvivoGen). THP1-Blue™ cells were seeded in 24-well plates and treated with PMA that induces the differentiation of the cells into macrophage-like cells, which are able to adhere and are primed for TLR signaling.

Before setting up the co-culture, the transepithelial electrical resistance (TEER) of the Caco-2 monolayers was measured (=oh time point). Transepithelial electrical resistance is the measurement of electrical resistance across a cellular monolayer and is a very sensitive and reliable method to confirm the integrity and permeability of the monolayer. The TEER of an empty insert was subtracted from all readings to account for the residual electrical resistance of an insert. Then, the Caco-2-bearing inserts were placed on top of the PMA-differentiated THP1-Blue™ cells. Briefly, the apical compartment (containing the Caco-2 cells) was filled with upper GIT suspensions diluted to 60% in cell culture medium. Upper GIT suspensions were centrifuged (5000 rpm; 5′), but not filter-sterilized. Four concentrations of the wood-based yeast were evaluated along with one concentration of the dextrose-based yeast. Cells were also treated apically with Sodium butyrate (NaB) (Sigma-Aldrich) as positive control. The basolateral compartment (containing the THP1-Blue™ cells) was filled with Caco-2 complete medium. Cells were also exposed to Caco-2 complete medium in both chambers as control. Cells were treated for 24 h, after which the TEER was measured (=24 h time point). After subtracting the TEER of the empty insert, all 24 h values were normalized to its own oh value (to account for the differences in initial TEER of the different inserts) and are presented as percentage of initial value.

TEER data were analyzed with a one-way analysis of variance (ANOVA) with Dunnett's multiple comparisons test. (*) represents statistically significant differences between the CM control and the treatments. (*), (**), (***) and (****) represent p<0.05, p<0.01, p<0.001 and p<0.0001, respectively.

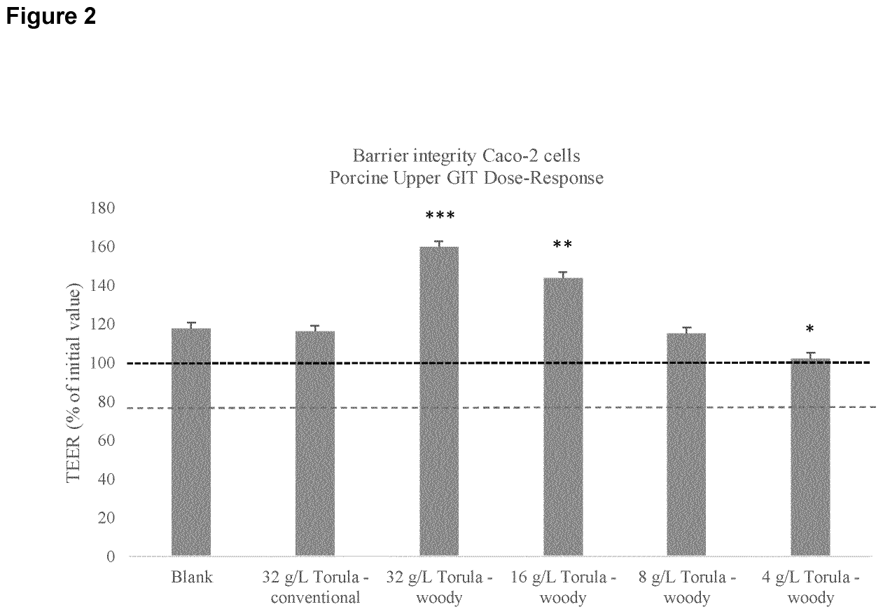

The CM line on FIG. 2 indicates the level of membrane disruption cause by PMA-activated THP-1 macrophages. Results of the experiment indicate that administration of torula yeast cultivated on wood hydrolysates improved the TEER across the membrane in a pig model in a dose-dependent manner (P<0.0001). The lowest wood-based yeast concentration negatively impacted TEER values. Yeast cultivated on dextrose was not significantly different from the blank (P>0.05): indicating that gastrointestinal permeability remained.

### Example 3: Administration of a Pure Product of Microorganisms Cultivated on Lignocellulosic Hydrolysates Improves the Transepithelial Electrical Resistance Across the Membrane

Dried, inactive torula yeast was cultivated on either wood-derived hydrolysates (Torula—woody) or on dextrose (Torula—conventional) in a 1,000 L bioreactor. Yeast was separated from the media in which it was cultivated, heat-inactivated, and dried to a moisture content of <10%. In one embodiment, the term “pure product” refers to a composition and/or microorganisms that has/have not been exposed to digestion.

The Caco-2 cell line is a human intestinal epithelial-like cell line with an extensive history of use as a model in mammalian gastrointestinal research. It is the most widely used cell line for developing human GIT in vitro models. Its use has been approved by the United States Food & Drug Administration as a surrogate for human absorption/permeability testing (FDA, 2017). Caco-2 cells may be grown to a confluent monolayer that provides a physical and biochemical barrier to the passage of ions and small molecules. At this stage, cells are differentiated and polarized in such way that, both morphologically and functionally, they resemble the enterocyte lining in the human small intestine. When these cells are co-cultured with PMA-activated THP-1 macrophages (i.e., a human monocytic cell line), THP-1-induced inflammation occurs and causes the disruption of the Caco-2 monolayer.

The Caco-2 co-culture experiment was performed as described in [4]. Briefly, Caco-2 cells (HTB-37; American Type Culture Collection) were seeded in 24-well semi-permeable inserts. Caco-2 monolayers were cultured for 14 days, with three medium changes/week, until a functional cell monolayer with a transepithelial electrical resistance (TEER) was obtained. Cells were maintained in Dulbecco's Modified Eagle Medium (DMEM) containing glucose and glutamine and supplemented with HEPES and 20% (v/v) heat-inactivated (HI) fetal bovine serum (FBS).

THP1-Blue™ (InvivoGen) cells were maintained in Roswell Park Memorial Institute (RPMI) 1640 medium containing glucose and glutamine, supplemented with HEPES, sodium pyruvate and 10% (v/v) HI-FBS. THP1-Blue™ are THP1 human monocytes stably transfected with a reporter construct expressing a secreted alkaline phosphatase (SEAP) gene under the control of a promoter inducible by the transcription factor nuclear factor kappa B (NF-κB). Upon TLR activation (e.g. by lipopolysaccharide (LPS); isolated from Gram-negative bacteria), NF-κB becomes activated and induces the expression and secretion of SEAP. SEAP activity can then be measured in the supernatants by using the QUANTI-Blue reagent (InvivoGen). THP1-Blue™ cells were seeded in 24-well plates and treated with PMA that induces the differentiation of the cells into macrophage-like cells, which are able to adhere and are primed for TLR signaling.

Before setting up the co-culture, the transepithelial electrical resistance (TEER) of the Caco-2 monolayers was measured (=oh time point). Transepithelial electrical resistance is the measurement of electrical resistance across a cellular monolayer and is a very sensitive and reliable method to confirm the integrity and permeability of the monolayer. The TEER of an empty insert was subtracted from all readings to account for the residual electrical resistance of an insert. Then, the Caco-2-bearing inserts were placed on top of the PMA-differentiated THP1-Blue™ cells. Briefly, the apical compartment (containing the Caco-2 cells) was filled with suspensions of the pure yeast product diluted to 60% in cell culture medium. Cells were also treated apically with Sodium butyrate (NaB) (Sigma-Aldrich) as positive control. The basolateral compartment (containing the THP1-Blue™ cells) was filled with Caco-2 complete medium. Cells were also exposed to Caco-2 complete medium in both chambers as control. Cells were treated for 24 h, after which the TEER was measured (=24 h time point). After subtracting the TEER of the empty insert, all 24 h values were normalized to its own oh value (to account for the differences in initial TEER of the different inserts) and are presented as percentage of initial value.

TEER data were analyzed with a one-way analysis of variance (ANOVA) with Dunnett's multiple comparisons test. (*) represents statistically significant differences between the CM control and the treatments. (*), (**), (***) and (****) represent p<0.05, p<0.01, p<0.001 and p<0.0001, respectively.

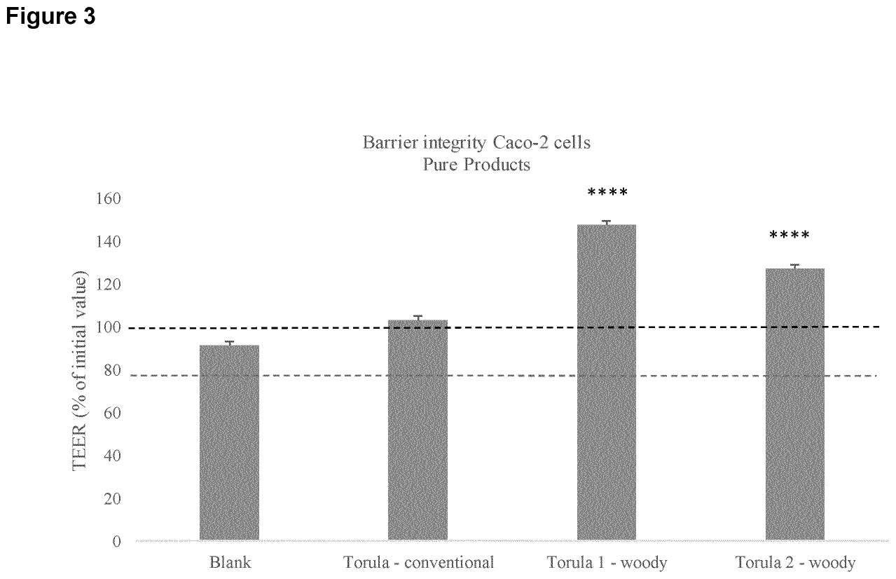

The CM line on FIG. 3 indicates the level of membrane disruption cause by PMA-activated THP-1 macrophages. Results of the experiment indicate that administration of torula yeast cultivated on wood hydrolysates as a pure product improved the TEER across the membrane to levels near those prior to membrane disruption (P<0.0001). Yeast cultivated on dextrose was not significantly different from the blank (P>0.05): indicating that gastrointestinal permeability remained.

### Example 4: Administration of Torula Yeast Cultivated on a Variety of Wood Hydrolysates as a Pure Product Improved the TEER Across the Membrane to Levels Above Those Prior to Membrane Disruption

Dried, inactive torula yeast was cultivated on various wood-derived hydrolysates in one of a 2-15,000 L bioreactor. The yeast products were cultivated on hydrolysates generated from varying fractionation technologies, summarized as: dilute acid hydrolysis, separation of C5 sugars (HH), followed by steam-explosion and enzymatic hydrolysis for C6 sugars (EHH); hemicellulose hydrolysis, followed by super-critical enzymatic hydrolysis (RED); dilute acid hydrolysis, then a second stage strong acid treatment and enzymatic hydrolysis (TBR); autohydrolysis, single stage steam-explosion pretreatment and enzymatic hydrolysis (BLU); autohydrolysis without acid addition, single state steam-explosion pretreatment and enzymatic hydrolysis (WHI); Combination of RED+EHH (REDEH); and extruder-based dilute acid hydrolysis and enzymatic hydrolysis (GRN). Yeast was separated from the media in which it was cultivated, heat-inactivated, and dried to a moisture content of <10%. A “pure product” refers to a composition and/or microorganisms that has/have not been exposed to digestion.

The Caco-2 cell line is a human intestinal epithelial-like cell line with an extensive history of use as a model in mammalian gastrointestinal research. It is the most widely used cell line for developing human GIT in vitro models. Its use has been approved by the United States Food & Drug Administration as a surrogate for human absorption/permeability testing (FDA, 2017). Caco-2 cells may be grown to a confluent monolayer that provides a physical and biochemical barrier to the passage of ions and small molecules. At this stage, cells are differentiated and polarized in such way that, both morphologically and functionally, they resemble the enterocyte lining in the human small intestine. When these cells are co-cultured with PMA-activated THP-1 macrophages (i.e., a human monocytic cell line), THP-1-induced inflammation occurs and causes the disruption of the Caco-2 monolayer.

The Caco-2 co-culture experiment was performed as described in [4]. Briefly, Caco-2 cells (HTB-37; American Type Culture Collection) were seeded in 24-well semi-permeable inserts. Caco-2 monolayers were cultured for 14 days, with three medium changes/week, until a functional cell monolayer with a transepithelial electrical resistance (TEER) was obtained. Cells were maintained in Dulbecco's Modified Eagle Medium (DMEM) containing glucose and glutamine and supplemented with HEPES and 20% (v/v) heat-inactivated (HI) fetal bovine serum (FBS).

THP1-Blue™ (InvivoGen) cells were maintained in Roswell Park Memorial Institute (RPMI) 1640 medium containing glucose and glutamine, supplemented with HEPES, sodium pyruvate and 10% (v/v) HI-FBS. THP1-Blue™ are THP1 human monocytes stably transfected with a reporter construct expressing a secreted alkaline phosphatase (SEAP) gene under the control of a promoter inducible by the transcription factor nuclear factor kappa B (NF-κB). Upon TLR activation (e.g. by lipopolysaccharide (LPS); isolated from Gram-negative bacteria), NF-κB becomes activated and induces the expression and secretion of SEAP. SEAP activity can then be measured in the supernatants by using the QUANTI-Blue reagent (InvivoGen). THP1-Blue™ cells were seeded in 24-well plates and treated with PMA that induces the differentiation of the cells into macrophage-like cells, which are able to adhere and are primed for TLR signaling.

Before setting up the co-culture, the transepithelial electrical resistance (TEER) of the Caco-2 monolayers was measured (=oh time point). Transepithelial electrical resistance is the measurement of electrical resistance across a cellular monolayer and is a very sensitive and reliable method to confirm the integrity and permeability of the monolayer. The TEER of an empty insert was subtracted from all readings to account for the residual electrical resistance of an insert. Then, the Caco-2-bearing inserts were placed on top of the PMA-differentiated THP1-Blue™ cells. Briefly, the apical compartment (containing the Caco-2 cells) was filled with suspensions of the pure yeast product diluted to 60% in cell culture medium. Cells were also treated apically with Sodium butyrate (NaB) (Sigma-Aldrich) as positive control. The basolateral compartment (containing the THP1-Blue™ cells) was filled with Caco-2 complete medium. Cells were also exposed to Caco-2 complete medium in both chambers as control. Cells were treated for 24 h, after which the TEER was measured (=24 h time point). After subtracting the TEER of the empty insert, all 24 h values were normalized to its own oh value (to account for the differences in initial TEER of the different inserts) and are presented as percentage of initial value.

TEER data were analyzed with a one-way analysis of variance (ANOVA) with Dunnett's multiple comparisons test. (*) represents statistically significant differences between the CM control and the treatments. (*), (**), (***) and (****) represent p<0.05, p<0.01, p<0.001 and p<0.0001, respectively.

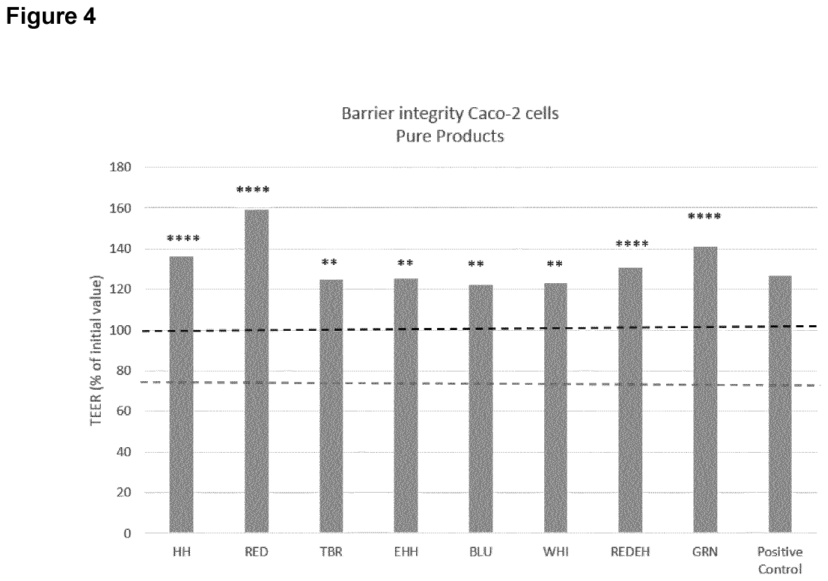

The CM line on FIG. 4 indicates the level of membrane disruption cause by PMA-activated THP-1 macrophages. Results of the experiment indicate that administration of torula yeast cultivated on a variety of wood hydrolysates as a pure product improved the TEER across the membrane to levels above those prior to membrane disruption (P<0.05).

### REFERENCES

[1] König, J., Wells, J., Cani, P. D., Garcia-Rödenas, C. L., MacDonald, T., Mercenier, A., Whyte, J., Troost, F., and Brummer, R. J. 2016. Human intestinal barrier function in health and disease. Clin. Transl. Gastroenter. 7: e196.
[2] Pluske, J. R., Turpin, D. L., and Kim, J. C. 2018. Gastrointestinal tract (gut) health in the young pig. Anim. Nutr. 4: 187-196.
[3] Roto, S. M., Rubinelli, P. M., and Ricke, S. C. 2015. An introduction to the avian gut microbiota and the effects of yeast-based prebiotic-type compounds as potential feed additives. Fron. Vet. Sci. 2(28): 1-18.
[4] Daguet, D., Pinheiro, I., Verhelst, A., Possemiers, S., and Marzorati, M. 2016. Arabinogalactan and fructooligosaccharides improve the gut barrier function in distinct areas of the colon in the Simulator of the Human Intestinal Microbial Ecosystem. J. Funct. Food. 20: 369-379.
[5] Mussatto, S. “Biomass Fractionation Technologies For a Lignocellulosic Feedstock Based Biorefinery.” (2016).

The features of the present invention disclosed in the specification, the claims, and/or in the accompanying figures may, both separately and in any combination thereof, be material for realizing the invention in various forms thereof.

1. A composition for use in a method of preventing and/or treating a gastrointestinal disorder, wherein said composition comprises a microorganism cultivated on or with lignocellulosic hydrolysate.

2-3. (canceled)

4. The composition according to claim 1, wherein said microorganism is a yeast selected from Candida sp., Saccharomyces sp., Kluyveromyces sp., Cyberlindnera sp., and combinations thereof; and/or
wherein said microorganism is a fungus selected from Aspergillus sp., Paecilomyces sp., Pleurotus sp., Trichoderma sp., and combinations thereof; and/or
wherein said microorganism is a bacterium selected from Bacillus sp. and Lactobacillus sp., and combinations thereof; and/or
wherein said microorganism is a microalgae selected from Spirulina sp. and Chlorella sp., and combinations thereof.

5. (canceled)

6. The composition according to claim 1 wherein said lignocellulosic hydrolysate is derived from biomass selected from forestry products and residues, agricultural residues, and waste products.

7-10. (canceled)

11. The composition according to claim 1, wherein said composition comprises a carrier selected from starches, proteins, and oils.

12. The composition according to claim 1, wherein said composition is formulated in the form of a powder, granules, a tablet, a pill, a capsule, a dispersion, a solution, a suspension, an emulsion, a liquid, a liquid concentrate, an oil mixture, a bar, a stick, a food product, a food supplement, a drink, a food formulation, a feed formulation, or a lozenge.

13. The composition according to claim 1, wherein said composition is a food formulation or a feed formulation comprising said composition in an amount of 0.001-50% by weight.

14. A method of preparing a composition of claim 1, comprising the following steps:
i) providing a microorganism and a lignocellulosic hydrolysate;
ii) culturing said microorganism using said lignocellulosic hydrolysate as a medium, thereby obtaining a mixture of microorganism and lignocellulosic hydrolysate;
iii) optionally, separating said microorganism from said medium;
iv) optionally, heat-inactivating said microorganism and/or said mixture;
v) optionally, drying said microorganism and/or said mixture;
vi) optionally, adding a carrier to said microorganism and/or said mixture;
vii) obtaining a composition comprising said microorganism and/or said mixture.

15. The method according to claim 14, wherein said providing said lignocellulosic hydrolysate comprises treating biomass using
biomass fractionation;
chemical breakdown;
enzymatic breakdown;
physical breakdown;
or a combination thereof.

16. The method according to claim 14, wherein said culturing comprises a cultivation of said microorganism on or with said lignocellulosic hydrolysate for a period of 0.1 to 600 h.

17. A method of preventing or treating a gastrointestinal disorder, and/or enhancing gastrointestinal health, and/or reducing gastrointestinal permeability, wherein said method comprises administering an effective amount of a composition, as defined in claim 1, to a patient in need thereof.

18. The method according to claim 17, wherein said disorder is selected from increased gastrointestinal permeability, leaky gut syndrome, inflammatory bowel disease, Crohn's disease, ulcerative colitis, irritable bowel syndrome, necrotic enteritis, colibacillosis, diarrhea/scours, porcine epidemic diarrhea virus, soybean meal induced enteritis, transient soy hypersensitivity, celiac disease, post-weaning diarrhea, necrotic enteritis, diabetes, rheumatoid arthritis, spondyloarthropathies, schizophrenia, cancer, fatty liver disease, atopy, and allergies.

19-21. (canceled)

22. The method according to claim 17, wherein said effective amount is in the range of from 0.001 to 50% by weight of a total weight of a daily food consumption.

23. The method according to claim 17, wherein said patient is a vertebrate.

24. The method according to claim 23,
wherein said vertebrate is selected from a
humans, pigs, dogs, cats,
chickens, turkeys, ducks,
salmon, trout, bass, bream, cod, catfish, tilapia,
shrimp, lobsters, crabs, octopuses, squid, oysters, clams, and mussels.

25. The method according to according claim 17, wherein said composition is administered to said patient via oral administration.

26. The method according to claim 17, wherein said composition further comprises lignocellulosic hydrolysate.

27. The method according to claim 17, wherein the microorganism of said composition is a yeast selected from Candida sp., Saccharomyces sp., Kluyveromyces sp., Cyberlindnera sp., and combinations thereof; and/or
wherein said microorganism is a fungus selected from Aspergillus sp., Paecilomyces sp., Pleurotus sp., Trichoderma sp., and combinations thereof; and/or
wherein said microorganism is a bacterium selected from Bacillus sp. and Lactobacillus sp., and combinations thereof; and/or
wherein said microorganism is a microalgae selected from Spirulina sp. and Chlorella sp., and combinations thereof.

28. The method according to claim 17, wherein said composition is formulated in the form of a powder, granules, a tablet, a pill, a capsule, a dispersion, a solution, a suspension, an emulsion, a liquid, a liquid concentrate, an oil mixture, a bar, a stick, a food product, a food supplement, a drink, a food formulation, a feed formulation, or a lozenge.

29. The method according to claim 17, wherein the patient is a human.
